(19) United States
(12) Patent Application Publication
(10) Pub. No.: US 2006/0182826 A1
(43) Pub. Date: Aug. 17, 2006

(54) Extracts of Asteraceae containing reduced levels of phototoxic thiophene derivatives and methods for preparing same

(75) Inventors: Elizabeth Barren, Kalamazoo, MI (US); Alexander R. Cazers, Richland, MI (US); Donald R. Berdahl, Lawton, MI (US)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: Kalamazoo Holdings, Inc., Kalamazoo, MI (US)

(21) Appl. No.: 11/352,166

(22) Filed: Feb. 10, 2006

Related U.S. Application Data
(60) Provisional application No. 60/652,478, filed on Feb. 11, 2005.

(51) Int. Cl. A61K 36/28

(52) U.S. Cl. 424/764

(57) ABSTRACT

The present invention is concerned with extracts of Asteraceae containing reduced levels of phototoxic thiophene derivatives. Novel methods that may result in about a fifty-fold to hundred-fold or more reduction of phototoxic thiophene levels in a commercially attractive process is described. The Asteraceae plant extracts comprising xanthophylls with reduced levels of thiophene derivatives may be used in applications that include nutritional supplements and cosmetics.

### FIELD OF THE INVENTION

The present invention is concerned with extracts of Asteraceae containing reduced levels of phototoxic thiophene derivatives. In particular, the genus Tagetes, which includes the familiar ornamental marigold varieties, is a major commercial source of lutein. Lutein and zeaxanthin have been identified as dietary components crucial to ocular health. In addition to beneficial carotenoids, Asteraceae plants are known to produce a family of phototoxic thiophene derivatives which are extracted along with the carotenoids in the preparation of marigold and related oleoresins. The presence of these phototoxic and contact dermatitis sensitizing compounds limits the usefulness of Tagetes extracts in nutritional supplement, cosmetic and eye health applications. Compositions and methods for preparing Asteraceae extracts, including Tagetes extracts, that are free from phototoxic thiophene derivatives or that contain these compounds at greatly reduced levels would be of significant commercial value.

### BACKGROUND OF THE INVENTION

Lutein and other carotenoids derived from plants of the family Asteraceae are important ingredients in the animal feed, nutritional supplement and cosmetics industries. In particular, the genus Tagetes, which includes the familiar ornamental marigold varieties, is a major commercial source of lutein (Antony, J. I. X. and Shankaranarayana, M. L., 2001, “Lutein: A Natural Colourant and a Phytonutrient for Eye Health Protection.” The World of Food Ingredients, April/May, 64-67). A mutant marigold variety, wherein zeaxanthin is the preponderant carotenoid, has also been developed and patented (U.S. Pat. No. 6,784,351 B2). Oleoresin extracts of marigold, provided in various states of refinement are well-known articles of commerce. Marigold flower petals or their extracts, in crude or refined form, are used as ingredients in animal feeds for the purpose of coloring the yolks of eggs and adding yellow pigmentation to the skin of poultry. Marigold extract has exempt color status only for this very limited use (21 CFR 73.295). At the time of this application, lutein may not be added legally to color human food in the United States. Lutein, including that derived from marigolds is, however, being used as a nutritional supplement and included in articles such as multivitamin preparations. FloraGLO® Lutein is a trademark of Kemin, Inc. Other commercial preparations of marigold oleoresins or refined marigold oleoresins are available from Cognis, Chrysantis, PIVEG and Kancor, Ltd.

Lutein and zeaxanthin are oxygenated (dihydroxy) carotene derivatives, also referred to as xanthophylls, and are present in the plant or in unrefined extracts largely in derivatized form as mono and diesters of fatty acids. Some free or non-esterified xanthophylls are often present as well in the native flowers and in their extracts.

Lutein and zeaxanthin have been identified as dietary components crucial to ocular health (Davies, N. P. and Morland, A. B. 2004, “Macular pigments: their characteristics and putative role.” Prog. Retinal Eye Res. 23, 533-59). Lutein and zeaxanthin are the two main pigments found in the human retina and there is increasing evidence that consumption of these pigments in sufficient amounts is a key factor in the prevention of age-related macular degeneration (AMD). These carotenoids are thought to protect the retina from oxidative damage through their role as antioxidants and light screeners. Zeaxanthin and lutein probably act through a number of mechanisms, including absorbing light, quenching highly reactive singlet molecular oxygen, and scavenging or reacting with free radicals. These are critical functions that serve to protect the eye from light-mediated damage.

In addition to beneficial carotenoids, Asteraceae plants are known to produce a family of phototoxic thiophene derivatives (Downum, K. R. and Wen, J., 1995, “The occurrence of photosensitizers among higher plants.” in Light Activated Pest Control, ACS Symposium Series 616, J. R. Heitz and K. R. Downum, eds., American Chemical Society, Washington, D.C., 135-143) that probably serve in nature as part of the plant's insect pest defense mechanism. These thiophene derivatives show high levels of phototoxicity in insects and other organisms (Arnason, J. T., Philogene, B. J. R., Morand, P., Imrie, K., Hasspieler, B. and Downe, A. E. R., 1989, “Naturally occurring and synthetic thiophenes as insecticides.” Amer. Chem. Soc. Symp. Ser. 387, 164-172). The phototoxic thiophenes are extracted along with the carotenoids in the preparation of marigold and related oleoresins. The most studied of these thiophene derivatives is α-terthiophene (α-terthienyl), which contains 3 thiophene rings. Bithiophene compounds are also present in these extracts. The bithiophene derivatives generally show less phototoxicity than α-terthiophene, but at least one bithiophene derivative, along with α-terthiophene, has also been identified as a contact dermatitis sensitizing agent (Hausen, B. M. and Helmke, B., 1995, “Butenylbithiophene, α-terthienyl and hydroxytremetone as contact allergens in cultivars of marigold (Tagetes sp.).” Contact Dermatitis 33, 33-37; Paulsen, E., 2002, “Contact sensitization from Compositae-containing herbal remedies and cosmetics.” Contact Dermatitis 47, 189-198; Rampone, W. M., McCullough, J. L., Weinstein, G. D., Towers, G. H. N., Berns, M. W. and Abeysekera, B. 1986, “Characterization of cutaneous phototoxicity induced by topical alpha-terthienyl and ultraviolet A radiation.” J. Invest. Dermat. 87(3), 354-357).

One of the major mechanisms of phototoxic action associated with thiophene derivatives involves the sensitized formation of radicals and highly reactive oxygen species, including singlet molecular oxygen (Boch, R., Mehta, B., Connoly, T., Durst, T., Arnason, J. T., Redmond, R. W. and Scaiano, J. C., 1996, “Singlet oxygen photosensitizing properties of bithiophene and terthiophene derivatives,” J. Photochem. Photobiol. A: Chem. 93, 39-47; Marles, R., Durst, T., Kobaisy, M., Coucy-Breau, C., Abou-Zaid, M, Arnason, J. T., Kacew, S., Kanjanapothi, D., Rujjanawate, C., Meckes, M. and Lozoya, X., 1995, “Pharmacokinetics, metabolism and toxicity of the plant-derived photoxin □-terthienyl.” Pharmacology and Toxicology 77, 164-168 and Nivsarkar, M., 1999, “Identification of alpha-terthienyl radical in vitro: a new aspect in alpha-terthienyl phototoxicity.” Current Science 76(10) 1391-1393). Since deactivation of reactive oxygen species, including singlet molecular oxygen is one of the proposed beneficial actions of lutein and zeaxanthin in the retina, the presence of phototoxic sensitizing agents in a nutritional supplement for eye health is clearly problematic. The presence of these phototoxic and contact dermatitis sensitizing compounds limits the usefulness of Tagetes extracts in nutritional supplement, cosmetic and eye health applications.

There is a perceived need in the marketplace for naturally derived xanthophylls that may serve as a dietary source in the form of a dietary supplement for healthful benefits. Significant levels of phototoxic thiophene derivatives in commercial preparations of zeaxanthin and lutein derived from Tagetes were observed using the methods described herein (see Example 3). There are no reports of xanthophyll preparations derived from Asteraceae with substantially reduced levels of thiophenes or methods to eliminate thiophene derivatives from the preparations. Therefore, the instant methods for preparing Asteraceae extracts, including Tagetes extracts, that are free from phototoxic thiophene derivatives or that contain these compounds at greatly reduced levels would be of significant commercial value.

Lutein products in the marketplace take several forms, including: simple extracts (oleoresins containing esterified xanthophyll pigments), purified lutein in the free-pigment form obtained through a saponification and concentration process, and purified lutein in the esterified form obtained through a pigment concentration process in the absence of a saponification step. An analysis of highly purified forms of lutein present in the market place shows that phototoxic thiophenes are largely absent from these preparations. The thiophenes have been removed, presumably, as an unintended consequence of the purification process. Standard purification processes such as crystallization and washing, either with or without a previous saponification step, have not been characterized as being intentionally used to remove or lower the concentration of these phototoxic compounds. U.S. Pat. No. 5,648,564 discloses treating an oleoresin marigold with caustic in the presence of propylene glycol, causing the lutein to be hydrolyzed to the free diol form. The free pigments precipitate out in solid form and may be separated and further purified by recrystallization, washing, or other processes well known in the art. U.S. Pat. No. 6,504,067 B1 discloses another saponification process for obtaining xanthophyll concentrates. U.S. Pat. No. 6,329,557 B1 discloses a process for obtaining highly purified concentrates from saponified marigold extracts that may have high levels of chlorophyll. These purified crystalline forms of the non-esterified xanthophyll are largely free of the thiophene compounds.

Crystallization and separation of solid pigments from a marigold oleoresin in the absence of a saponification step leads to a fraction in esterified form with higher xanthophyll concentration than the starting oleoresin and a fraction with lower xanthophyll concentration. It is expected that the enriched fraction would have a lower phototoxic thiophene to xanthophyll ratio than the liquid fraction. The precipitation of carotenoid pigments may be assisted using solvents. U.S. Pat. No. 6,737,535 discloses a process using ketonic solvents, whereas U.S. Pat. No. 6,191,293 B1 discloses a process using alcoholic solvents.

It should be noted that forms of lutein (or zeaxanthin in the case of the mutant marigold) more like the native extract may be desired. Many consumers of nutritional supplements believe that the more complicated native mixtures benefit from synergistic interactions between their various components and favor minimal processing of natural extracts. Studies have shown that purified or synthetic carotenoids seem to be a less effective way of obtaining the compound's putative beneficial effects than the consumption of equivalent amounts through food sources (Khachik, F., Beecher, G. R. and Smith J. C., 1995, “Lutein, Lycopene, and Their Oxidative Metabolites in Chemo-prevention of Cancer”, J. Cell. Biochem., Suppl. 22, 236-246 and Mordi, R., 1993, “Carotenoids: functions and degradation.” Chem. Ind. (February), 79-83). This implies that a process that purifies the main carotenoid constituent to a great extent may have a detrimental effect on any synergistically enhanced activity that might have been originally present in the native or whole extract. The saponification/purification methods available to the art are clearly not suitable for producing whole extracts which preserve the natural spectrum of ingredients but which also have greatly reduced levels of phototoxic thiophenes.

### OBJECTS OF THE INVENTION

It is an object of the present invention to provide novel Tagetes extracts with nutritionally desirably levels of xanthophylls in predominantly esterified form, but with greatly reduced levels of phototoxic and contact dermatitis sensitizing thiophene compounds.

It is an object of the present invention to provide novel methods for preparing Asteraceae extracts, including Tagetes extracts, that are free from phototoxic thiophene derivatives or that contain these compounds at greatly reduced levels.

It is an object of the present invention to optimize the method of preparing Asteraceae extracts, including Tagetes extracts, that are free from phototoxic thiophene derivatives or that contain these compounds at greatly reduced levels, by identifying specific forms of carbon, specific solvents and other specific conditions that may result in about a fifty-fold to hundred-fold or more reduction of phototoxic thiophene levels.

### DESCRIPTION OF THE INVENTION

Levels of phototoxic terthiophene derivatives in Asteraceae plant extracts may be dramatically lowered by treating the extracts with certain forms of carbon (charcoal), or extracting the carotenoid source while contacting it with an effective type and amount of carbon. The degree to which the phototoxic thiophene derivatives are removed, and the degree to which the desired xanthophyll pigments are retained, depends on the type and amount of carbon used, upon the type and amount of solvent used and upon the contact time. Permutations, such as optimizing the temperature, performing multiple treatments, lengthening or shortening the contact time, adjusting the particle size of the carbon, activating the carbon, and the like, are within the knowledge of one skilled in the art and these optimizations are considered a part of the present disclosure.

Marigold extracts may be treated with specific types of carbon to greatly reduce the levels of phototoxic thiophene compounds that occur naturally in them. Marigold extracts (oleoresins) with reduced amounts of phototoxic thiophene derivatives may be prepared by carrying out the extraction of the plant material (e.g. marigold petals or meal) in the presence of certain forms of adsorptive carbon.

### BRIEF DESCRIPTION OF THE INVENTION

What we therefore believe to be comprised by our invention may be summarized inter alia in the following words:

A method of preparing Asteraceae plant extracts exhibiting reduced levels of thiophene derivatives comprising the steps of contacting a solution of an Asteraceae extract with a particulate form of carbon, separating the carbon from the extract solution and removing the solvent from the solution; such a

method wherein the Asteraceae extract is a Tagetes extract; such a

method wherein the Asteraceae extract contains xanthophylls; such a

method wherein the Asteraceae extract is in the form of a miscella; such a

method wherein the thiophene derivatives comprise alpha-terthiophene and butenylbithiophene; such a

method wherein the carbon is an activated carbon; such a

method wherein the carbon is packed in the form of a bed; such a

method wherein the Asteraceae extract solution is passed through the bed of particulate carbon; such a

method wherein the solvent is selected from ethanol, methanol, isopropyl alcohol, acetone, hexanes, cyclohexane, methyl ethyl ketone, methyl-t-butyl ether, methylene chloride, chloroform, tetrahydrofuran, ethyl acetate, supercritical carbon dioxide, subcritical carbon dioxide, liquid propane, subcritical propane supercritical propane, liquid hydrofluorocarbons, subcritical hydrofluorocarbons, supercritical hydrofluorocarbons, and mixtures thereof; such a

method of preparing Asteraceae plant extracts exhibiting reduced levels of thiophene derivatives, comprising the steps of contacting Asteraceae plant matter with a particulate form of carbon admixed in a solvent, separating the insoluble solids and removing the solvent from the solution; such a

method wherein the plant matter comprises flower petals; such a

method wherein the solvent is selected from ethanol, methanol, isopropyl alcohol, acetone, hexanes, cyclohexane, methyl ethyl ketone, methyl-t-butyl ether, methylene chloride, chloroform, tetrahydrofuran, ethyl acetate, supercritical carbon dioxide, subcritical carbon dioxide, liquid propane, subcritical propane supercritical propane, liquid hydrofluorocarbons, subcritical hydrofluorocarbons, supercritical hydrofluorocarbons, and mixtures thereof; such a

method for reducing the level of thiophene derivatives in Asteraceae oleoresin comprising the steps of contacting an Asteraceae oleoresin with a solvent, contacting the resulting mixture with a particulate form of carbon, separating the carbon from the mixture after a period of time and removing the remaining solvent from the solution; such a

Asteraceae extract which is an oleoresin comprising less than about 15 micrograms of thiophenes per milligram of xanthophylls; such a

Asteraceae oleoresin comprising less than about 7 micrograms of thiophenes per milligram of xanthophylls; such a

Asteraceae oleoresin comprising less than about 0.3 micrograms of thiophenes per milligram of xanthophylls.

### BRIEF DESCRIPTION OF THE FIGURES

FIG. 1 represents HPLC chromatograms as set forth in Example 1 of an alpha terthiophene standard (trace A) and a commercial marigold oleoresin (trace B). The 350 nm profile peak at a retention time of 5.13 minutes in both traces corresponds to an alpha terthienyl species.

FIG. 2 represents the UV spectral comparison of commercial marigold oleoresin major peaks as recorded in the HPLC analysis of Example 1, in comparison to the UV spectra of an alpha terthiophene standard.

FIG. 3 represents an HPLC chromatogram as set forth in Example 2 of a commercial marigold oleoresin.

FIG. 4 represents a graph of HPLC data area counts of terthiophene concentration as measured over contact time with activated carbon as set forth in Example 5.

### DETAILED DESCRIPTION OF THE INVENTION

Tagetes oleoresins may be treated to dramatically lower the concentration of phototoxic ingredients by dissolving them in a solvent, treating the mixture with carbon, removing the carbon after a period of time, and removing the solvent to reconstitute the oleoresin. While there is some loss of carotenoid pigments using this process, if the appropriate carbon and solvent is used, removal of thiophene derivatives is maximized and loss of carotenoids minimized.

Asteraceae (Tagetes) oleoresins with reduced levels of phototoxic agents are conveniently prepared by extracting plant matter with a solvent or solvent mixture, treating the resulting miscella with an amount of an effective carbon, filtering the treated miscella to remove the carbon and evaporating the solvent to form an oleoresin with lowered level of phototoxic thiophene derivatives. The types of solvents that may be used are well known in the art and include hexanes, acetone, methyl ethyl ketone, isopropyl alcohol, ethanol, methanol, chlorinated hydrocarbons, liquid or superfluid propane, subcritical or supercritical carbon dioxide, and the like. Alternately, the solvent or solvent mixture used to form the initial miscella may be removed and replaced in varying amounts with another solvent or solvent mixture. This plant extract, now in a second solvent or solvent mixture, may be treated with an effective carbon, and filtered to remove the carbon. Removal of the second solvent or solvent mixture provides an oleoresin with reduced levels of phototoxic ingredients.

Additionally, Asteraceae (Tagetes) oleoresins with reduced levels of phototoxic thiophene derivatives may be produced by solvent extracting a mixture of plant matter and an effective carbon, as described in Example 8.

Certain commercially available carbon products are more effective than others. Example 6 shows the results of an investigation of 15 commercial carbon samples. The carbons tested were:

| | |
|---|---|
| Norit ® PAC 200 | Norit ® MAG 301 |
| Norit ® SX Ultra | Darco ® KBG |
| Darco ® S-51HF | Darco ® KB |
| ADP-PULV | Norit ® CASPF |
| CARBOCHEM ® P-1000 | Norit ® CN1 |
| Darco ® S-51 | Darco ® KB-B |
| Darco ® 12X20LI | Norit ® KB-FF |
| APA 12X40 | |

Norit ® is a registered trademark of American Norit Company
Darco ® is a registered trademark of American Norit Company
CARBOCHEM ® is a registered trademark of Carbochem, Inc.
APA is manufactured by Calgon Carbon Co.

Six of the tested carbons were shown to have the best performance. These are Norit® PAC 200, Norit® SX Ultra, Darco® S-51HF, ADP-PULV, CARBOCHEM® P-1000 and Darco® S-51.

TABLE 1
The effect of varying the amount of carbon used was
determined in experiments described in Example 6.

| | | % Thiophenes |
|---|---|---|
| Carbon Type | Amount Added (grams) | Removed |
| Norit ® PAC 200 | 0.11 | 93 |
| | 0.10 | 95 |
| | 0.07 | 89 |
| | 0.03 | 69 |
| | 0.02 | 50 |
| Norit ® SX Ultra | 0.11 | 92 |
| | 0.10 | 94 |
| | 0.07 | 87 |
| | 0.03 | 62 |
| | 0.02 | 51 |
| Darco ® S-51HF | 0.11 | 92 |
| | 0.10 | 90 |
| | 0.07 | 82 |
| | 0.03 | 52 |
| | 0.02 | 36 |
| ADP-PULV | 0.11 | 91 |
| | 0.10 | 91 |
| | 0.07 | 83 |
| | 0.03 | 58 |
| | 0.02 | 42 |
| CARBOCHEM ® P-1000 | 0.11 | 92 |
| | 0.10 | 91 |
| | 0.07 | 83 |
| | 0.03 | 53 |
| | 0.02 | 42 |
| Darco ® S-51 | 0.11 | 89 |
| | 0.10 | 90 |
| | 0.07 | 81 |
| | 0.03 | 51 |
| | 0.02 | 40 |
| Darco ® 12X20LI | 0.11 | 68 |
| APA 12X40 | 0.11 | 66 |
| Norit ® MAG 301 | 0.11 | 59 |
| Darco ® KBG | 0.11 | 47 |
| Darco ® KB | 0.11 | 47 |
| Norit ® CASPF | 0.11 | 44 |
| Norit ® CN1 | 0.11 | 32 |
| Darco ® KB-B | 0.11 | 26 |
| Norit ® KB-FF | 0.11 | 26 |

A wide range of solvents are used in this process; however, the results of Example 4 show that not all solvents are equally effective media for the adsorption of α-terthiophene. Toluene, for example, is shown to be a rather poor medium for this process. Food-grade solvents, meeting government regulations for the manufacture of oleoresins, are particularly useful in the disclosed process. These include ethanol, methanol, isopropyl alcohol, acetone, hexanes, cyclohexane, methyl ethyl ketone, methylene chloride, chloroform, tetrahydrofuran and ethyl acetate. Chlorinated solvents are not preferred due to environmental concerns. Supercritical or subcritical carbon dioxide; liquid, subcritical or supercritical propane; liquid, subcritical or supercritical hydrofluorocarbons may also be used. In some cases mixtures of solvents may give superior results.

The particle size of the particulate carbon may be adjusted, by one skilled in the art, to optimize the thiophene removal process. Carbon preparations range from finely powdered to granulated, with amounts and particle size being easily optimized by simple experiment.

Contact time has also been found to be an important parameter in the removal of thiophene derivatives using carbon. Example 5 describes experiments showing the importance of optimizing the contact time.

The carbon may be dispersed in a liquid mixture of solvent and Asteraceae extract. In another embodiment, the carbon may be packed in the form of a bed and the solvent/Asteraceae mixture moved through the bed to effect removal of the thiophene derivatives.

Oleoresins prepared by the instant method are superior to un-treated extracts in the treatment and prevention of ocular diseases and have associated advantages in their use as nutritional supplements. Extracts purified by the instant methods may be further treated to yield pigment concentrates by methods described in U.S. Pat. No. 5,648,564, U.S. Pat. No. 6,504,067 B1, U.S. Pat. No. 6,329,557 B1, U.S. Pat. No. 6,737,535 or U.S. Pat. No. 6,191,293 B1.

### EXAMPLES

The present invention will be better understood in connection with the following examples, which are intended as an illustration of and not a limitation on the scope of the invention.

Discussion of Analytical Methodology

Analytical evaluations for the marigold extracts encompass two target analyte groups, the xanthophylls and the thiophenes. The xanthophylls are evaluated using a relatively long gradient HPLC Method-B of Example 2 that permits adequate separation of the free, mono-, and di-esterfied xanthophylls. Although this method also yields the thiophene species profile, a more time efficient method of Example 1, HPLC Method-A, is also presented that is useful for monitoring the thiophene removal efforts.

### Example 1

### HPLC Method-A

This external standard method specifically targets the thiophene group of analytes, to the exclusion of the xanthophylls. It is a rapid and accurate way to monitor the thiophene content of marigold oleoresins as well as to track their subsequent removal by the processes described herein. The oleoresin was initially dissolved in a suitable organic solvent such as hexane, ethyl acetate or methyl tert-butyl ether. A typical concentration, depending upon the xanthophyll content and/or the oleoresin solubility characteristics, was approximately 20 mg oleoresin per mL solvent. This analysis solution was processed by the following instrumental method:
Reagents are ACS grade, and the solvents are HPLC grade. A Waters HPLC 2695 separations module wass configured with mobile phase components of 5 mM ammonium acetate (A) and acetonitrile (B). The HPLC column consists of two 5 cm by 2.1 mm Zorbax XDB C18 columns in series, with a Phenomenex C18 Securityguard™ cartridge. The column temperature was set to 40° C. The mobile phase composition was isocratic with 35% A and 65% B. The flow rate was 0.4 mL per minute. The detector was a Waters 2996 photodiode array (PDA) set to scan from 220 nm to 460 nm. Typical injection volume was 2 μL. The run time is 7.0 minutes.

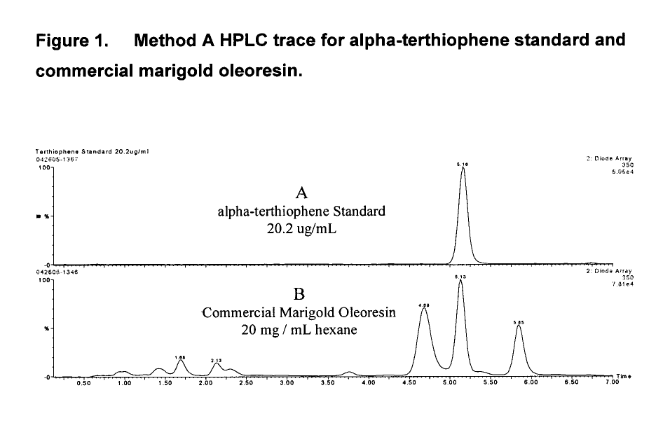

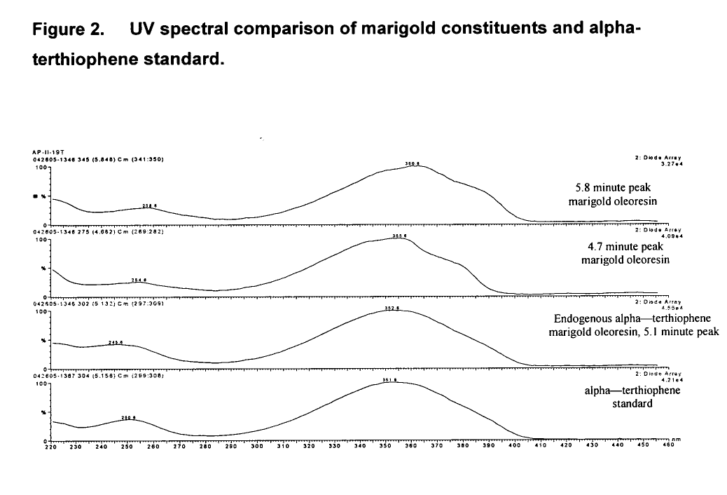

The marigold oleoresin profile revealed the presence of several peaks with similar UV spectra. The 350 nm profile peak at a retention time of 5.13 minutes (FIG. 1B) was the apparent alpha terthienyl species, having the same retention time and UV spectra as the synthetic standard material (2,2:5′,2″-terthiophene, Sigma No. 311073) (FIG. 1A). The other two peaks in the marigold oleoresin profile at retention times 4.7 minutes and 5.9 minutes are unknowns with very similar UV spectra (FIG. 2) to alpha-terthienyl and may be the known marigold phototoxins butenylbithiophene and its hydroxylated analog (Arroo, R. R. J., De Brouwer, A. P. M., Croes, A. F., and Wullems, G. J., (1995), “Thiophene interconversion in elicitor-treated toots of Tagetes patula L.”, Plant Cell Reports, 15, 133-137). Since the latter two species exhibit similar UV absorption, chromatographic polarity and charcoal adsorption kinetics, they were included in the quantitative sum as a thiophene peak envelope along with alpha-terthienyl.

Alpha-terthienyl is a major phototoxic species in genus Tagetes and is also commercially available in pure form. Therefore, this compound was used as a marker for evaluating the endogenous thiophene removal processes.

An alpha-terthienyl calibration curve was generated by preparing appropriate standard solutions in ethyl acetate, acetone, hexane or methyl tertbutyl ether, across a range of approximately 0.5 μg/mL to 50 μg/mL. The calibration curve is linear with a correlation coefficient of 0.999. Subsequent experimental solutions should be diluted to the appropriate volume so that the maximum peak area of the marker compound is within the absolute area counts of the highest calibration curve standard solution area.

### Example 2

### HPLC Method-B

This method generates qualitative and quantitative profiles of both the thiophenes and the xanthophylls in the marigold oleoresins. Sample preparation and the HPLC instrumentation was the same as for Method A of Example 1, except that the PDA was set to scan from 210 nm to 690 nm. The HPLC profiles were viewed at 350 nm wavelength, which yielded a useful profile of the thiophenes as well as the xanthophylls. The mobile phase components were 5 mM ammonium acetate (A), methyl tert-butyl ether (B), and acetonitrile (C). The method used the following gradient:

| Time | A % | B % | C % | Flow | Curve |
|---|---|---|---|---|---|
| 0.00 | 98.0 | 0.0 | 2.0 | 0.200 | 1 |
| 20.00 | 30.0 | 0.0 | 70.0 | 0.300 | 6 |
| 25.00 | 5.0 | 0.0 | 95.0 | 0.300 | 8 |
| 40.00 | 5.0 | 70.0 | 25.0 | 0.400 | 6 |
| 41.00 | 5.0 | 0.0 | 95.0 | 0.400 | 6 |
| 42.00 | 5.0 | 0.0 | 95.0 | 0.400 | 1 |
| 43.00 | 98.0 | 0.0 | 2.0 | 0.400 | 1 |
| 50.00 | 98.0 | 0.0 | 2.0 | 0.400 | 1 |
| 54.00 | 98.0 | 0.0 | 2.0 | 0.400 | 1 |
| 55.00 | 98.0 | 0.0 | 2.0 | 0.200 | 1 |

The resulting profile revealed two general regions of interest. The thiophene elution region was apparent at approximately 24-26 minutes and the xanthophylls eluted between 27 and 41 minutes. The minor peaks at about 29-31 minutes and about 33-36.5 minutes, were the non-esterified and mono-esterified lutein esters, respectively, along with some minor additional related species. The major elution band between about 37 minutes and 41 minutes were the cis and trans lutein di-esters.

An alpha-terthiophene calibration curve was generated along the lines of Method-A of Example 1, utilizing the same range of concentrations. A second calibration curve was constructed using free lutein (Xanthophyll, Sigma No. X-6250) across a range of approximately 40 μg/ml to 1330 μg/ml, using a suitable solvent such as hexane, ethyl acetate or methyl tert-butyl ether.

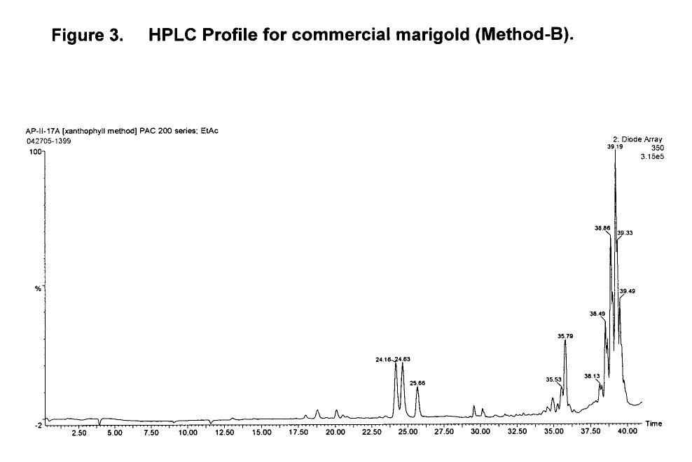

A linear calibration curve was obtained with a correlation coefficient of 0.999. The 350 nm profile was generated (FIG. 3) and the peaks within the 27 minute to 41 minute retention time region were integrated and this area sum was then utilized for the subsequent xanthophylls concentration calculations.

### Example 3

### Determining α-terthiophene (α-terthienyl) Levels in Commercial Sources of Zeaxanthin and Lutein by GC-EI-MS and GC-PFPD

The analyses were performed on a Varian 3800 gas chromatograph in-line with a Saturn 2000 ion trap mass spectrometer. The mass spectrometer was operated in the electron ionization mode with scanning from 40 u to 650 u. The NIST Standard Reference Database, version 1.6 was used for peak identification. The GC-pulsed flame photometric detector was configured for sulfur-specific detection as per vendor specification. Data acquisition utilized the Varian Saturn GC/MS data station (v5.51). Gas chromatography was performed on a Supelco MDN-5S fused silica capillary column, 30 m×0.25 mm i.d., 0.25 um film (p/n 24384)). The column flow rate was 1.5 ml helium/minute; the injector temperature was 240° C.; the detector temperature was 230° C.; the oven temperature program was 120° C. to 260° C. at 8° C./minute, hold at 260° C. for 4.5 minutes; the injector split ratio was 1 for PFPD analysis and 20 for the GC-EI-MS runs. The injection volume was 0.5 μL. A PFPD calibration curve for α-terthienyl (Aldrich, 2,2′:5′,2″-Terthiophene, #311073, 99% purity) from 160 ng per ml to 5000 ng per ml acetone was generated and used for subsequent quantitation. Several commercial marigold oleoresins and two nutritional supplement capsules were tested. The respective oleoresins were dissolved in acetone at 3300 microgram of oleoresin per ml acetone prior to injection. The resulting sample area response was converted to the corresponding α-terthienyl ppm value from the calibration curve by solving the second order polynomial equation that was generated by a curve-fitting algorithm using Microsoft Excel 2000. The results are listed below, with the limit of detection (LOD)=1.0 microgram/capsule for the capsules.

TABLE 2
Concentrations of α-terthiophene in commercial
extracts and nutritional supplements.

| Sample | α-terthiophene concentration | |
|---|---|---|
| Commercial Zeaxanthin Capsule 1 | 1.4 | microgram/capsule |
| Commercial Lutein Capsule 2 | 2.2 | microgram/capsule |
| Commercial Marigold Extract 1 | 515 | ppm |
| Commercial Marigold Extract 2 | 1150 | ppm |
| Commercial Marigold Extract 3 | 760 | ppm |

### Example 4

### Showing the Effect of Solvent on the Adsorption of α-Terthiophene by NORIT® PAC 200 in a Model System

α-Terthiophene standard solutions were made up in a variety of HPLC grade solvents, including methyl-t-butyl ether (MTBE), methanol (MeOH), hexane, and toluene with a concentration of 40 ppm. Aliquots (2×5 mL) of standard solutions were shaken on a gyroshaker for 30 min at room temperature with varying amounts (0-0.10 g) of NORIT® PAC 200 carbon. After shaking, the carbon was in contact with the standard solutions at room temperature for approximately 2 hours (total contact time 2.5 hours). Then 1.5 mL aliquots of the solutions were centrifuged and the resulting supernatant was analyzed by HPLC-UVNIS at 350 nm, using Method-A, as described in Example 1. The results are listed in Table 3.

TABLE 3
The Adsorption of α-Terthiophene by
NORIT ® PAC 200: Effect of Solvent.

| Hα-Terthiophene | | α- | % α- |
|---|---|---|---|
| Standard Solution | NORIT ® | Terthiophene, | Terthiophene |
| Type | PAC 200, g | ppm* | Adsorbed |
| Methanol | 0.0000 | 39.7 | 0 |
| Methanol | 0.0144 | 0.2 | >99 |
| Methanol | 0.0244 | <dl** | >99 |
| Methanol | 0.0667 | <dl | >99 |
| Methanol | 0.0913 | <dl | >99 |
| Methyl-t-butyl ether | 0.0000 | 44.5 | 0 |
| Methyl-t-butyl ether | 0.0146 | 0.5 | 98.8 |
| Methyl-t-butyl ether | 0.0248 | <dl | >99 |
| Methyl-t-butyl ether | 0.0646 | <dl | >99 |
| Methyl-t-butyl ether | 0.1015 | <dl | >99 |
| Toluene | 0.0000 | 36.6 | 0 |
| Toluene | 0.0135 | 34.2 | 14 |
| Toluene | 0.0229 | 32.2 | 19 |
| Toluene | 0.0651 | 25.6 | 35 |
| Toluene | 0.1000 | 22.7 | 42 |
| Hexane | 0.0000 | 30 | 0 |
| Hexane | 0.0154 | 2 | 94 |
| Hexane | 0.0246 | <dl | 99 |
| Hexane | 0.0386 | <dl | >99 |
| Hexane | 0.1168 | <dl | >99 |

*Standard α-terthiophene hexane solutions ranging from 0.35-44 ppm were used to generate a standard curve and the following linear regression was used to calculate the concentration α-terthiophene, y = 372.36x + 285.43.
**The detection limit (dl) was ˜0.2 ppm.

### Example 5

### Showing the Effect of Contact Time on Thiophene-Derivative Removal

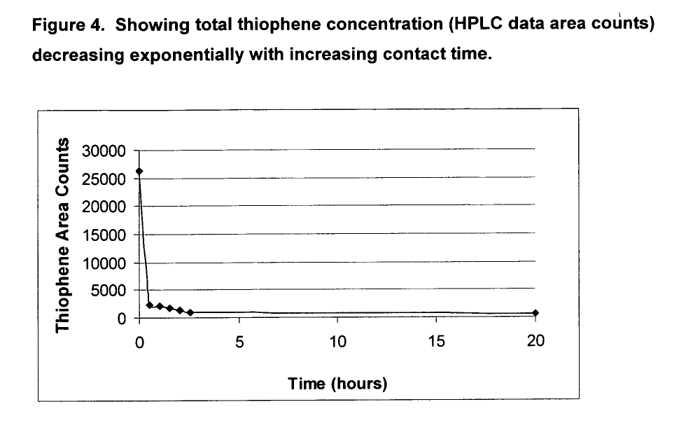

A 20 mg/mL Synthite MRY1004 marigold oleoresin solution was prepared in HPLC grade hexane. NORIT® PAC 200 carbon (0.0770 g) was then stirred with a 10 mL aliquot of Synthite MRY1004 marigold oleoresin solution at room temperature. Then 1.5 mL aliquots were removed from solution at times: 0, 30, 60, 90, 120, 150, and 1200 min and centrifuged. The resulting supernatant was analyzed by HPLC-UVNIS (350 nm) using Method-A of Example 1, and a time dependence graph was generated, as shown in FIG. 4. The removal is very rapid, giving roughly a 10-fold reduction in 30 minutes and about a 100-fold reduction in 150 minutes.

### Example 6

### Showing the Performance of a Variety of Commercially Available Carbon Products on Reducing the Level of Thiophene Derivatives from a Commercial Marigold Oleoresin

A 2 g/100 mL Scitech® marigold oleoresin solution was prepared in HPLC grade ethyl acetate (EtOAc). Aliquots (2×5 mL) of the Scitech® marigold oleoresin solution were shaken on the gyroshake for 60 min at room temperature with various commercial carbons (0.11 g). After shaking, 1.5 mL aliquots of the solutions were centrifuged and the resulting supernatants were analyzed by HPLC-UVNIS at 350 nm, using Method A, described above. Quantification of the thiophenes was made using α-terthiophene standard solutions. Of the fifteen carbons investigated, the six most absorptive were further examined in a dose-response study. Fresh 2 g/100 mL Scitech® marigold oleoresin solution in EtOAc solution was prepared and aliquots (2×5 mL) of this solution were shaken with varying amounts of the six carbons for 1 hour at room temperature. Lastly, 1.5 mL aliquots of the solutions were centrifuged and the resulting supernatant was analyzed by HPLC-UVNIS at 350 nm, again using Method-A of Example 1. The results are shown in Table 1, in the body of the specification. Six of the tested carbons were shown to have the best performance. These are Norit® PAC 200, Norit® SX Ultra, Darco® S-51 HF, ADP-PULV, CARBOCHEM® P-1000 and Darco® S-51.

### Example 7

### NORIT® PAC 200 Effects on Thiophene and Xanthophyll Levels in Scitech® Marigold Oleoresin Hexane Solution

A 2 g/100 mL Scitech® marigold oleoresin solution was prepared in HPLC grade hexane. Aliquots (2×5 mL) of the Scitech® marigold oleoresin solution were shaken on the gyroshake for 60 min at room temperature with various amounts of NORIT® PAC 200. After shaking, 1.5 mL aliquots of the solutions were centrifuged and the resulting supernatants were analyzed by HPLC-UVNIS at 350 nm, using Method-B of Example 2. Standard solutions of α-terthiophene in HPLC grade hexane and xanthophyll in HPLC grade methyl-t-butyl ether were made. Each set of standard solutions was analyzed by HPLC-UVNIS at 350 nm. Calibration curves were generated and the resulting linear regressions were used to calculate the concentration of total thiophenes and xanthophyll of all of the samples, the results are summarized in Table 4. The data shows about a 100-fold reduction in terthiophene levels with a negligible reduction in xanthophyll concentration. A useful measure of marigold oleoresin quality is captured in the ratio of terthiophene to xanthophylls, measured in micrograms of terthiophene to milligrams of xanthophyll pigments.

TABLE 4
Thiophene and Xanthophyll Dose Response
using NORIT ® PAC 200.

| NORIT ® PAC | [Terthiophene], | [Xanthophyll], | μg terthiophene/ |
|---|---|---|---|
| 200, g | μg/ml* | mg/ml** | mg xanthophyll |
| 0.0000 | 69.8 | 2.68 | 26 |
| 0.0202 | 22.3 | 2.92 | 7.6 |
| 0.0300 | 13.7 | 2.72 | 5 |
| 0.0707 | 1.93 | 2.57 | 0.75 |
| 0.1012 | 0.69 | 2.60 | 0.27 |

*Standard α-terthiophene hexane solutions (40.4-0.505 ppm) were used to generate a standard curve and the following linear regression was used to calculate the concentration of total thiophenes, y = 399.11x + 373.55.
**Standard xanthophyll methyl-t-butyl ether solutions (0.040-1.334 mg/mL) were used to generate a standard curve and the following linear regression was used to calculate the xanthophyll concentration, y = 70.9829x − 32.5262.

Scitech® is a registered trademark of Qingdao Scitech Perfume Co. Ltd.

### Example 8

### Marigold Extraction in the Presence of NORIT® PAC 200

Fresh marigold petals were dehydrated and ground in preparation for extraction. Extracts were prepared in a 1:10:100 (w/w/w) of NORIT® PAC 200 carbon:dried ground marigold:HPLC grade solvent (either ethyl acetate or hexane) and stirred for one hour. Control extracts were also prepared without carbon. Two successive extractions of each sample were completed. The resulting mixtures were filtered and the filtrates combined and concentrated by rotary evaporation. Finally the extracts were taken up in solvent and analyzed for thiophene and xanthophyll content by HPLC-UVNIS at 350 nm, using Method-B of Example 2. The results are listed in Table 5.

TABLE 5
Adsorption of Thiophene and Xanthophyll by
NORIT ® PAC 200 in Bulk Extraction.

| | Thiophene | | | |
|---|---|---|---|---|
| | Peak | % Thiophene | Xanthophyll | % Xanthophyll |
| Sample ID | Area | Adsorbed | Peak Area | Adsorbed |
| Ethyl Acetate | 58981 | 0.0 | 283659 | 0.0 |
| Control | | | | |
| Ethyl Acetate | 1208 | 97.95 | 188761 | 33.4550 |
| with Carbon | | | | |
| Hexane Control | 77929 | 0.0 | 321200 | 0.0 |
| Hexane with | <dl | <dl | 173123 | 46.1012 |
| Carbon | | | | |

1. A method of preparing Asteraceae plant extracts exhibiting reduced levels of thiophene derivatives comprising the steps of contacting a solution of an Asteraceae extract with a particulate form of carbon, separating the carbon from the extract solution and removing the solvent from the solution.

2. The method of claim 1, wherein the Asteraceae extract is a Tagetes extract.

3. The method of claim 1, wherein the Asteraceae extract contains xanthophylls.

4. The method of claim 1, wherein the Asteraceae extract is in the form of a miscella.

5. The method of claim 1, wherein the thiophene derivatives comprise alpha-terthiophene and butenylbithiophene.

6. The method of claim 1, wherein the carbon is an activated carbon.

7. The method of claim 1, wherein the carbon is packed in the form of a bed.

8. The method of claim 7, wherein the Asteraceae extract solution is passed through the bed of particulate carbon.

9. The method of claim 1, wherein the solvent is selected from ethanol, methanol, isopropyl alcohol, acetone, hexanes, cyclohexane, methyl ethyl ketone, methyl-t-butyl ether, methylene chloride, chloroform, tetrahydrofuran, ethyl acetate, supercritical carbon dioxide, subcritical carbon dioxide, liquid propane, subcritical propane supercritical propane, liquid hydrofluorocarbons, subcritical hydrofluorocarbons, supercritical hydrofluorocarbons, and mixtures thereof.

10. A method of preparing Asteraceae plant extracts exhibiting reduced levels of thiophene derivatives, comprising the steps of contacting Asteraceae plant matter with a particulate form of carbon admixed in a solvent, separating the insoluble solids and removing the solvent from the solution.

11. The method of claim 10, wherein the plant matter comprises flower petals.

12. The method of claim 10, wherein the solvent is selected from ethanol, methanol, isopropyl alcohol, acetone, hexanes, cyclohexane, methyl ethyl ketone, methyl-t-butyl ether, methylene chloride, chloroform, tetrahydrofuran, ethyl acetate, supercritical carbon dioxide, subcritical carbon dioxide, liquid propane, subcritical propane supercritical propane, liquid hydrofluorocarbons, subcritical hydrofluorocarbons, supercritical hydrofluorocarbons, and mixtures thereof.

13. A method for reducing the level of thiophene derivatives in Asteraceae oleoresin comprising the steps of contacting an Asteraceae oleoresin with a solvent, contacting the resulting mixture with a particulate form of carbon, separating the carbon from the mixture after a period of time and removing the remaining solvent from the solution.

14. An Asteraceae extract resulting from the method of claim 1, which is an oleoresin comprising less than about 15 micrograms of thiophenes per milligram of xanthophylls.

15. The Asteraceae oleoresin of claim 14, comprising less than about 7 micrograms of thiophenes per milligram of xanthophylls.

16. The Asteraceae oleoresin of claim 14, comprising less than about 0.3 micrograms of thiophenes per milligram of xanthophylls.
